(19) United States
(12) Patent Application Publication
(10) Pub. No.: US 2010/0113869 A1
(43) Pub. Date: May 6, 2010

(54) SYSTEMS AND METHODS FOR TREATING ANTERIOR PELVIC ORGAN PROLAPSE

(76) Inventor: Ian L. Goldman, Scottsdale, AZ (US)

Correspondence Address: SQUIRE SANDERS & DEMPSEY LLP, TWO RENAISSANCE SQUARE, 40 NORTH CENTRAL AVENUE, SUITE 2700, PHOENIX, AZ, 85004-4498, US

(21) Appl. No.: 12/430,828

(22) Filed: Apr. 27, 2009

Related U.S. Application Data
(60) Provisional application No. 61/095,223, filed on Nov. 3, 2008.

(51) Int. Cl. A61F 2/00

(52) U.S. Cl. 600/37

(57) ABSTRACT

A system for treating anterior pelvic organ prolapse according to one aspect of the present invention comprises a cape for supporting an anterior organ (such as a bladder) that has prolapsed into a vagina, a plurality of support arms coupled to the cape, and a plurality of fasteners for attaching the support arms to a portion of the pelvic wall. Each one of the plurality of fasteners is coupled to a respective one of the plurality of support arms. The system further includes a tension adjustment system. Among other things, the present invention provides an integrated, pre-fabricated solution to anterior pelvic organ prolapse. All components of the system can thus be quickly deployed, without the need to build the repair system piecemeal. Among other things, this results in quicker surgeries, less risk to the patient from anesthesia, and less risk of failure of the repair due to the improper assembly of the repair system. Additionally, the present invention provides a completely transvaginal solution to treating anterior pelvic organ prolapse, is easier for less experienced surgeons to perform, and poses far less risk of injury or death to the patient.

### CROSS-REFERENCE TO RELATED APPLICATION

This application claims priority to U.S. Provisional Patent Application Ser. No. 61/095,223, filed Nov. 3, 2008, the disclosure of which is incorporated by reference in its entirety for all purposes.

### DESCRIPTION OF THE INVENTION

1. Field of the Invention

The present invention relates to systems and methods for treating anterior pelvic organ prolapse, and more particularly, to transvaginal anterior pelvic organ prolapse treatment utilizing an integrated, light-weight support system.

2. Background of the Invention

Pelvic organ prolapse (POP) is one of the most common conditions requiring urogynecological surgery. POP occurs when the muscles of the pelvic floor are unable to support the pelvic organs (such as the bladder). Cystocoele, or bladder prolapse, is a common form of anterior POP whereby the bladder drops and pushes against the walls of the vagina. Cystocoele commonly occurs when the muscles of the pelvic floor are damaged or weakened during childbirth.

According to an analysis of U.S. procedure codes for prolapse surgery, it is estimated that more than a half-million procedures are performed annually accounting for more than one-billion health care dollars. Epidemiological studies demonstrate that only a fraction of women with POP seek medical attention and that the incidence (i.e., the number of new cases during a specified time) and prevalence (i.e., the number of affected persons during a specified time divided by the number of persons in the population at that time) rates based on surgical intervention significantly underestimate the magnitude of this problem.

It is estimated that women have an 11% lifetime risk of undergoing a single operation for POP and Urinary Incontinence by age 80 years. Studies have shown that 50% of parous women lose pelvic floor support resulting in POP. Although difficult to determine the number of women affected by POP, it is estimated that the general population of women will increase by 22% over the next 30 years and the demand for care for pelvic floor disorders increase by 45%.

Surgery for pelvic floor disorders such as POP and Stress Urinary Incontinence (SUI) is aimed at restoring or improving the function of the pelvic organs. The approach to these complex problems has varied over time and with the advancements in anatomical knowledge, technology and the development of biological and synthetic materials. One conventional transvaginal approach for repairing cystocoele involves suturing the edges of the vaginal wall over the cystocoele to keep it in place. This approach suffers from a high failure rate, particularly in the first five years after the surgery, since it entirely relies on the integrity of the weakened tissue/muscle of the vaginal walls that allowed the cystocoele to occur in the first place.

Another conventional method for repairing a cystocoele involves a combined transvaginal and transobturator approach. In this method, four incisions are made outside the patient's vagina on the vulvar skin. Helical needles are inserted through the incisions, through the obturator foramen in the pelvis, and finally through the vaginal wall. The arms of a mesh cape placed within the vagina are attached to the ends of the needles, and the needles are withdrawn, bringing the arms of the cape outside the patient's body to be tightened. This method is extremely time consuming and difficult. It is difficult, particularly for inexperienced surgeons, to ensure the needles pierce the pelvic sidewalls in the correct location without causing injury to the patient. The patient is also at risk from infection, bleeding, nerve damage, and from the effects of being under anesthesia for long periods of time during the procedure.

As such, conventional systems and methods for addressing POP are still complicated, invasive and have a significant potential for harm to the patient, including death. The present invention addresses these and other issues.

### SUMMARY OF THE INVENTION

Methods and systems according to the present invention can be used in treating all forms of anterior wall defects, including cystocoele. The repair of cystocoele in accordance with the present invention is significantly quicker than other conventional approaches. For example, a surgeon employing systems and methods of the present invention can repair a cystocoele in about fifteen minutes, where other methods can take in excess of an hour. Additionally, the present invention provides a completely transvaginal solution to treating anterior pelvic organ prolapse, is easier for less experienced surgeons to perform, and poses far less risk of injury or death to the patient.

A system for treating anterior pelvic organ prolapse according to one aspect of the present invention comprises a cape for supporting an anterior organ that has prolapsed into a vagina, a plurality of support arms coupled to the cape, and a plurality of fasteners for attaching the support arms to a portion of a pelvic sidewall. Each one of the plurality of fasteners is coupled to a respective one of the plurality of support arms. The system may further include a tension adjustment system. Among other things, the present invention provides an integrated, pre-fabricated solution to cystocoele repair. All components of the system can thus be quickly deployed, without the need to build the repair system piecemeal. Among other things, this results in quicker surgeries, less risk to the patient from anesthesia, and less risk of failure of the repair due to the improper assembly of the repair system. The multiple points of fixation also help recreate and restore the normal structural support of the pelvic sidewall.

A method for treating anterior pelvic organ prolapse according to another aspect of the present invention comprises attaching a first pair of support arms to portions of the pelvic sidewall, the first pair of support arms coupled to a cape. The method further includes positioning the cape over a bladder that has prolapsed into a vagina. The method also includes attaching a second pair of support arms to a Levator Ani Muscle of the pelvic sidewall, the second pair of support arms coupled to the cape. The method further includes adjusting the first and second pairs of support arms using a tension adjustment system. This method can be performed more quickly (and is less difficult) than conventional solutions for treating anterior pelvic organ prolapse, allowing less experienced surgeons to perform the repair without the risk of substantial harm to the patient present in conventional methods.

Both the foregoing summary and the following detailed description are exemplary and explanatory only and are not restrictive of the invention.

### BRIEF DESCRIPTION OF THE DRAWINGS

A more complete understanding of the present invention may be derived by referring to the detailed description and claims when considered in connection with the following illustrative figures.

FIG. 1 depicts an exemplary system for treating anterior pelvic organ prolapse according to various aspects of the present invention.

FIGS. 2A-2E depict exemplary fasteners for attaching a support arm to portions of the pelvic sidewall according to various aspects of the present invention.

FIG. 3 is a flow diagram depicting an exemplary method for anterior organ prolapse treatment according to various aspects of the present invention.

### DETAILED DESCRIPTION OF EXEMPLARY EMBODIMENTS

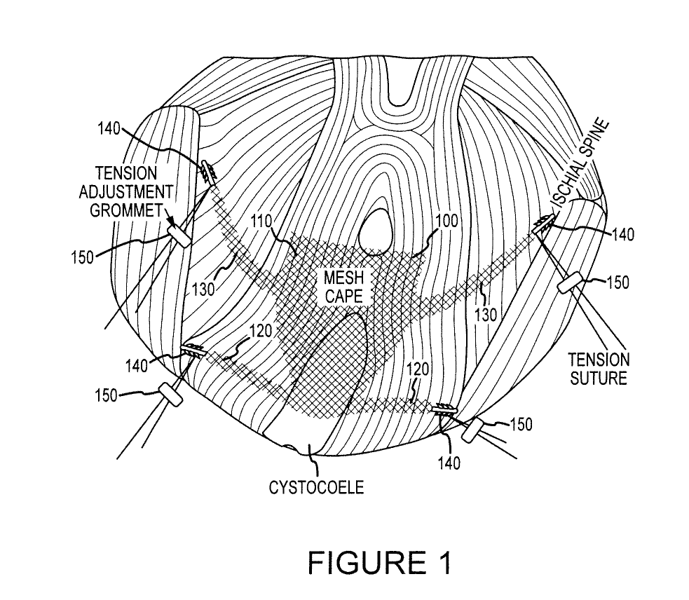

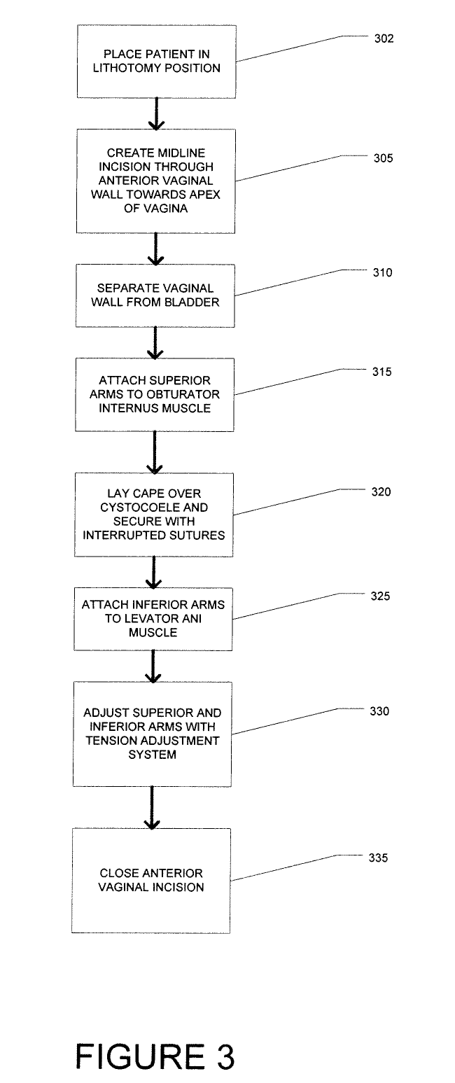

An exemplary system 100 according to various aspects of the present invention is depicted in FIG. 1. This system 100 may be used in conjunction with the method described in FIG. 3, as well as with any subset or combination of the elements thereof. The system 100 shown in FIG. 1 may also be used in conjunction with any other suitable embodiments of systems and methods, including systems and methods for treating other forms of pelvic organ prolapse (POP).

System 100

The exemplary system 100 depicted in FIG. 1 includes a cape 110, a pair of superior support arms 120, a pair of inferior support arms 130, fasteners 140 coupled to the support arms 120, 130, and a tension adjustment system 150. The system 100 is shown covering a cystocoele as viewed from the top of the patient's pelvis. The support arms 120, 130 attach to the pelvic sidewalls to keep the system 100 in place in order to retain the cystocoele. As used herein, the term “pelvic sidewall” may refer to any tissue, muscle, ligament, or other structure surrounding and/or defining the vaginal cavity, including the Obturator Internus muscle, Levator Ani muscle, ischial spine, and/or vaginal mucosa.

Cape 110

The cape 110 is configured to support an anterior organ (such as a bladder) that has prolapsed into the vagina. The cape 110 may be any size, shape, and configuration to allow the cape 110 to cover and support the prolapsed organ. The cape 110 may be formed from any suitable material, such as a biological material (including porcine dermus) and/or synthetic material (including polypropylene). In the present exemplary embodiment, the cape 110 is formed from a lightweight synthetic mesh. The mesh cape 110 allows the tissue of the pelvic sidewall to grow across the cape, strengthening the area of the prolapse while the cape retains the bladder. The cape 110 can be partially flexible and/or partially elastic to aid in the placement of the system and/or retention of the bladder. The size and shape of the cape 110 in FIG. 1 can be altered (e.g., by a surgeon using scissors) prior to installation to conform to a cystocoele having a particular size and/or shape. Additionally, the cape 110 may include materials of relatively less rigidity and/or flexibility than other portions of the cape 110 to, for example, provide strength and stability to the system 100.

Support Arms 120 130

The support arms 120, 130 attach to the walls of the vagina to help secure the cape 110 in place in order to retain the prolapsed bladder. The support arms 120, 130 can be any desired dimension (including size, shape, length, width, and thickness). The dimensions between different support arms 120, 130, as well as the dimensions throughout an individual support arm 120, 130, may vary. For example, referring to the exemplary system 100 depicted in FIG. 1, the width of the inferior support arms 130 can be wider at their proximal ends (i.e., where they attach to the cape 120) than at their distal ends (i.e., where they attach to their respective fasteners 140). Additionally, the superior support arms 120 are shorter than the inferior support arms 130 to allow the superior support arms 120 to attach to portions of the pelvic sidewall that are farther from the cystocoele than the portions of the pelvic sidewall to which the inferior support arms 130 attach. In the present exemplary embodiment, a pair of superior support arms 120 are coupled to opposing sides of the cape, while a pair of inferior support arms 130 are coupled to opposing sides of the cape. Using the fasteners 140 described below, the superior support arms 120 are configured for attachment to the Obturator Internus muscle of the pelvic sidewall, while the inferior support arms 130 are configured for attachment to the Levator Ani muscle of the pelvic sidewall.

The support arms 120, 130 can be formed from any desired material. The support arms 120, 130 can be made from the same, or different material(s) than the cape 110. For example, the support arms 120, 130 can be formed from synthetic material (such as polypropylene), biological material (such as porcine dermus), or a combination of synthetic and biological materials. The support arms 120, 130 and cape 110 can be made from a unitary piece of material, such as a piece of synthetic and/or biological mesh. Among other things, forming the cape 110 and the support arms 120, 130 from a unitary piece of material can increase the strength and/or flexibility of the system 100. Additionally, the unitary construction of the system 100 allows the system 100 to be quickly deployed, without the need to assemble the system 100 piecemeal. Among other things, this results in quicker surgeries, less risk to the patient from anesthesia, and less risk of failure of the repair due to the improper assembly of the repair system 100.

Fasteners 140

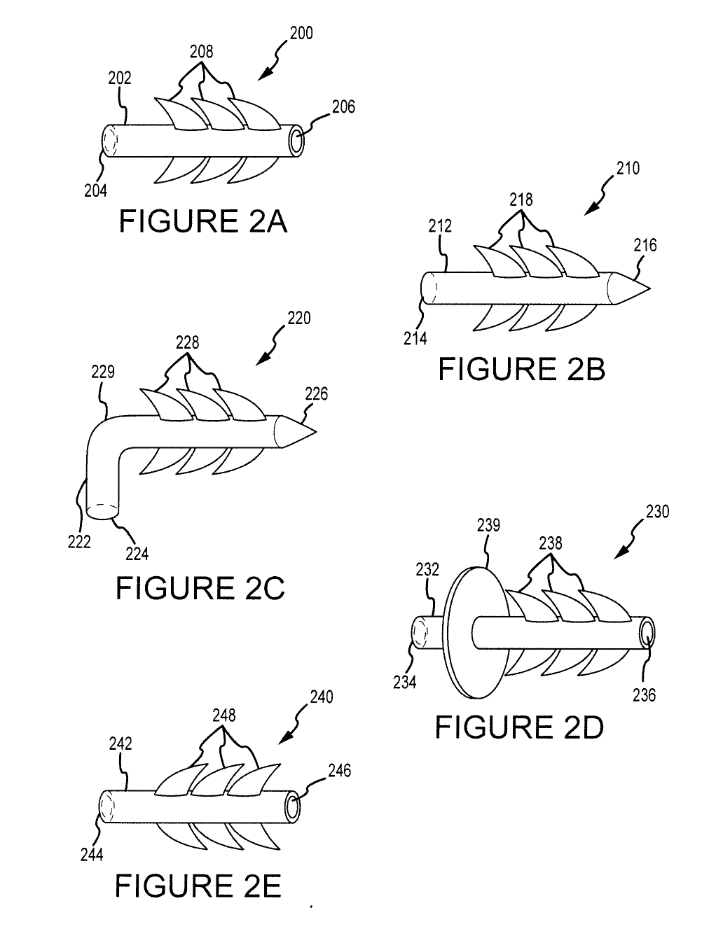

Fasteners 140 coupled to the distal ends of the support arms 120, 130 are configured to attach the support arms 120, 130 to the pelvic sidewall. Any suitable fastener may be used in conjunction with the present invention to attach the support arms 120, 130 to any suitable portion of the pelvic sidewall. FIG. 2A depicts one exemplary embodiment of a fastener according to various aspects of the present invention. In FIG. 2A, a fastener 200 comprises a body portion 202, a proximal end 204 that is coupled to a support arm (not shown), a distal end 206, and one or more barbs 208 coupled to the exterior of the body portion 202 for attaching the fastener to the pelvic sidewall. The body portion 202 is cylindrical, approximately 1 to 2 centimeters long, and approximately 0.25 centimeters in diameter in the exemplary embodiment depicted in FIG. 2A, though the body portion of a fastener utilized in conjunction with the present invention may be any suitable size and shape. Likewise, the exemplary fasteners depicted in FIGS. 2B-2D and discussed below may also be any suitable size and shape.

The body portion 202 of the exemplary fastener 200 is hollow to allow a surgeon to pass a needle through the body portion 202 and perforate the pelvic sidewall in order to insert the fastener 200 in the perforation. The needle can then be withdrawn. The barbs 208 help keep the fastener 200 attached to the pelvic sidewall (such as in the Obturator Internus muscle or Levator Ani muscle) once inserted in the perforation. To assist in inserting the fastener 200 in the perforation, the distal end 206 may be tapered or comprise any other shape or configuration to help guide the fastener 200 into the perforation. In the exemplary fastener 200 depicted in FIG. 2A, the barbs extend from the exterior of the body portion 202 towards the proximal end 204 in order to resist being pulled free from the pelvic sidewall.

FIG. 2B depicts an alternate exemplary fastener 210 according to various aspects of the present invention. In this exemplary embodiment, fastener 210 includes a body portion 212 having a proximal end 214 that is coupled to a support arm (not shown), a pointed distal end 216, and one or more barbs 218. This exemplary embodiment allows a surgeon to perforate the pelvic sidewall with the pointed distal end 216, which obviates the need to perforate the pelvic sidewall with a separate needle. The body portion may be at least partially hollow (e.g., to allow a tool to be inserted within the body portion 212 to help press the fastener 210 into place) or solid (e.g., to provide added rigidity and/or strength).

FIG. 2C depicts yet another exemplary fastener 220 according to various aspects of the present invention. In this exemplary embodiment, fastener 220 includes a body portion 222 having a proximal end 224 that is coupled to a support arm (not shown), a pointed distal end 226, and one or more barbs 228. In this exemplary embodiment, the body portion comprises a non-rectilinear section 229. In this embodiment, the non-rectilinear section 229 is curved to create ninety degree angle to allow the proximal end 224 to run parallel to the pelvic sidewall. Among other things, the non-rectilinear section 229 aids a surgeon in keeping the support arm coupled to the fastener 220 closer to the surface of the pelvic sidewall. Instead of protruding out from the surface of the pelvic sidewall, the support arm (connected to the proximal end 224) can be run from the site of the fastener 220 to the cape with less of a gap between the pelvic sidewall and the support arm.

The exemplary fastener 230 depicted in FIG. 2D includes a hollow body portion 232 having a proximal end 234 that is coupled to a support arm (not shown), a distal end 236, and one or more barbs 238. Alternately, this embodiment could implement a pointed distal end (such as ends 216 and 226 shown in FIGS. 2B and 2C, respectively). The exemplary embodiment depicted in FIG. 2D also includes a structure 239 to prevent the fastener from penetrating the pelvic sidewall beyond a predetermined depth. The structure may be any size, shape and configuration, and may be located anywhere on the fastener to prevent it from penetrating beyond a predetermined depth in the pelvic sidewall. In FIG. 2D, the structure comprises a lip radiating from the external surface of the body portion 234. In this embodiment, the lip 239 extends beyond the total outside diameter of the barbs 238 to allow it to contact the pelvic sidewall, preventing the fastener 230 from penetrating beyond the depth of the lip 239.

The exemplary fastener 240 depicted in FIG. 2E includes a hollow body portion 242 having a proximal end 244 that is coupled to a support arm (not shown), a distal end 246, and one or more barbs 248. In this exemplary embodiment, the barbs 248 extend from the external surface of the body portion towards the distal end 296 (i.e., opposite to the barbs 208 shown in FIG. 2A). Among other things, this allows a surgeon to more easily remove the fastener 240 (e.g., to correct its placement), while still allowing the barbs 248 to engage the muscle or tissue of the pelvic sidewall to keep the fastener 240 in place once the fastener 240 is correctly located.

Any of the features of the exemplary fasteners depicted in FIGS. 2A-2E and described above may be combined or substituted in accordance with the present invention. While the body portions of the fasteners are depicted as cylindrical, alternate embodiments may include body portions having any size, shape, and configuration. For example, the body portions may be at least partially flexible and/or non-rectilinear. Likewise, the barbs utilized on the exemplary fasteners may be any size, shape, and configuration to allow the fastener to attach to the pelvic sidewall. Fasteners used in conjunction with the present invention can be permanently, or releasably, coupled to the support arms 120, 130. Any of the barbs used in conjunction with the fasteners depicted in FIGS. 2A-2E can be configured to dynamically collapse and extend from the exterior of the body portion to allow the fastener to be inserted and removed from the pelvic sidewall.

Tension Adjustment System 150

The tension adjustment system 150 allows a surgeon to adjust the tension of the support arms 120, 130 and/or the cape 110 to ensure the cape 110 properly retains the cystocoele. The tension of the support arms 120, 130 and/or cape 110 may be adjusted in any manner using any desired system or device. In the present exemplary embodiment, each support arm 120, 130 includes a tension adjustment system 150 that comprises a grommet coupled to the support arm using a tensioning suture. The tensioning suture loops through an aperture in the support arm. In the case that the support arms are formed from mesh, the aperture through which the tensioning suture loops may be one of the openings formed by the mesh itself. The tensioning grommet includes two holes through which the two respective ends of the tensioning suture pass. A surgeon can adjust the tensioning grommet along the tensioning suture with one hand while holding the two ends of the tensioning suture with the other hand. Once the desired tension is achieved in the support arm and/or cape, the ends of the tensioning suture can be tied, holding the grommet in place and maintaining the level of tension. In this manner, a surgeon is able to increase or decrease the amount of tension in the support arms 120, 130 or cape 110 in order to ensure an anterior organ (such as the bladder) that has prolapsed into the vagina is properly supported.

The grommets depicted in FIG. 1 are substantially cylindrical, though grommets in alternate embodiments of the present invention can be any size, shape, and configuration. The grommets can be formed from any material, such as a biocompatible synthetic. Likewise, the tensioning suture can be formed from any material and can be any suitable length and diameter to help maintain the tension of the support arm.

Any other tension adjustment system may be used in conjunction with the present invention to adjust the tension of the support arms 120, 130 and/or the cape 110. In other exemplary embodiments, the tensioning system 150 for each support arm can be formed from any other suitable fastener connected to the support arm, such as a button, clasp, buckle, hook and loop fastener, clip, ring, clamp, snap, and/or staple. In one such embodiment, a button comprising a first portion that is attached to a support arm has a pair of openings through which a tensioning suture is looped. A second portion of the button is releasably attached to the first portion, the second portion also including a pair openings through which the two ends of the tensioning suture can be passed. A surgeon can adjust the tension in the support arm, fasten the first and second portions of the button together, then tie the tensioning suture to maintain the level of tension in the support arm.

Operation

An exemplary method according to various aspects of the present invention is depicted in FIG. 3. This method may be practiced in conjunction with the system depicted in FIG. 1, as well as the fasteners depicted in FIGS. 2A-2E. In this method, the patient is placed in the lithotomy position (302) and a midline incision is made through the anterior vaginal wall towards the apex of the vagina (305). The vagina is separated from the bladder (310) and the superior arms coupled to the cape of the cystocoele repair system are attached to the Obturator Internus muscle on opposing sides of the cystocoele (315). The cape is laid over the cystocoele and secured with interrupted sutures (320). The inferior arms coupled to the cape of the cystocoele repair system are attached to the Levator Ani muscle approximately ½ centimeter from to the respective ischial spines on opposing sides of the cystocoele (325). The superior and inferior arms of the cystocoele repair system are adjusted and tightened with the tension adjustment system (330) (e.g., by using a tension adjustment grommet secured with a tension suture). The anterior vaginal incision is closed (335) and the procedure terminated.

The particular implementations shown and described above are illustrative of the invention and its best mode and are not intended to otherwise limit the scope of the present invention in any way. Indeed, for the sake of brevity, conventional data storage, data transmission, and other functional aspects of the systems may not be described in detail. Methods illustrated in the various figures may include more, fewer, or other steps. Additionally, steps may be performed in any suitable order without departing from the scope of the invention. Furthermore, the connecting lines shown in the various figures are intended to represent exemplary functional relationships and/or physical couplings between the various elements. Many alternative or additional functional relationships or physical connections may be present in a practical system.

Changes and modifications may be made to the disclosed embodiments without departing from the scope of the present invention. These and other changes or modifications are intended to be included within the scope of the present invention, as expressed in the following claims.

What is claimed is:

1. A system for treating anterior pelvic organ prolapse, the system comprising:
a cape for supporting an anterior organ that has prolapsed into a vagina;
a plurality of support arms coupled to the cape; and
a plurality of fasteners for attaching the support arms to a portion of a pelvic sidewall, wherein each one of the plurality of fasteners is coupled to a respective one of the plurality of support arms.

2. The system of claim 1, wherein the cape is at least partially flexible.

3. The system of claim 2, wherein the cape comprises a mesh.

4. The system of claim 3, wherein the mesh is formed from a synthetic material.

5. The system of claim 4, wherein the synthetic material includes polypropylene.

6. The system of claim 3, wherein the mesh is formed from a biological material.

7. The system of claim 6, wherein the biological material includes porcine dermus.

8. The system of claim 1, wherein the anterior organ is a bladder.

9. The system of claim 1, wherein the cape and the support arms are formed from a unitary piece of material.

10. The system of claim 9, wherein the cape and support arms comprise a mesh.

11. The system of claim 10, wherein the mesh is formed from polypropylene.

12. The system of claim 10, wherein the mesh is formed from porcine dermus.

13. The system of claim 1, wherein the cape and the plurality of support arms are formed from the same material.

14. The system of claim 13, wherein the cape and the plurality of support arms are formed from a polypropylene mesh.

15. The system of claim 13, wherein the cape and the plurality of support arms are formed from a porcine dermus mesh.

16. The system of claim 1, wherein the plurality of support arms comprise:
a pair of superior support arms for attachment to an Obturator Internus muscle of the pelvic sidewall; and
a pair of inferior support arms for attachment to a Levator Ani muscle of the pelvic sidewall.

17. The system of claim 16, wherein the pair of superior support arms are coupled to opposing sides of the cape, and wherein the pair of inferior support arms are coupled to opposing sides of the cape.

18. The system of claim 1, wherein each of the plurality of fasteners comprises:
a body portion comprising:
an exterior;
a proximal end coupled to one of the plurality of support arms; and
a distal end; and
one or more barbs coupled to the exterior of the body portion for attaching the fastener to the portion of the pelvic sidewall.

19. The system of claim 18, wherein at least one of the plurality of fasteners is releasably coupled to one of the plurality of support arms.

20. The system of claim 18, wherein the one or more barbs extend from the exterior of the body portion towards the proximal end of the body portion.

21. The system of claim 18, wherein the one or more barbs extend from the exterior of the body portion towards the distal end of the body portion.

22. The system of claim 18, wherein the one or more barbs are configured to dynamically collapse and extend from exterior of the body portion to allow the fastener to be inserted and removed from the pelvic sidewall.

23. The system of claim 18, wherein the body portion is at least partially flexible.

24. The system of claim 18, wherein at least part of the body portion is non-rectilinear.

25. The system of claim 18, wherein the body portion is hollow and the proximal end of the body portion and the distal end of the body portion are open to allow a needle to pass through the body portion.

26. The system of claim 18, wherein the distal end of the body portion is pointed for piercing the pelvic sidewall.

27. The system of claim 18, further comprising a structure coupled to the body portion for preventing the fastener from penetrating the pelvic sidewall beyond a predetermined depth.

28. The system of claim 27, wherein the structure comprises a lip extending from the exterior of the body portion.

29. The system of claim 1, further comprising a tension adjustment system.

30. The system of claim 29, wherein the tension adjustment system comprises a plurality of grommets, wherein each one of the plurality of grommets is coupled to a respective one of the plurality of support arms using a respective one of a plurality of tension sutures.

31. The system of claim 30, wherein each of the plurality of grommets comprise one or more openings to allow the passage of the respective tension suture therethrough.

32. The system of claim 29, wherein the tension adjustment system comprises a plurality of fasteners, wherein each one of the plurality of fasteners is coupled to a respective one of the plurality of support arms using a respective one of a plurality of tension sutures.

33. The system of claim 32, wherein the plurality of fasteners include a button, wherein each button comprises:
a first portion attached to the respective support arm and comprising one or more openings to allow the passage of the respective tension suture therethrough; and
a second portion releasably attached to the first portion and comprising one or more openings to allow the passage of the respective tension suture therethrough.

34. A method for treating anterior pelvic organ prolapse, the method comprising:
attaching a first pair of support arms to portions of a pelvic sidewall, the first pair of support arms coupled to a cape;
positioning the cape over an anterior organ that that has prolapsed into a vagina; and
attaching a second pair of support arms to other portions of the pelvic sidewall, the second pair of support arms coupled to the cape.

35. The method of claim 34, further comprising:
creating a midline incision through an anterior portion of a wall of the vagina;
separating the vaginal wall from the anterior organ; and
closing the midline incision.

36. The method of claim 34, further comprising attaching the cape to the pelvic sidewall using interrupted sutures.

37. The method of claim 34, wherein the anterior organ is a bladder.

38. The method of claim 34, wherein the first pair of support arms are attached to the Levator Ani Muscle approximately ½ centimeters from an Ischial Spine.

39. The method of claim 34, wherein the first pair of support arms are coupled to opposing sides of the cape, and wherein the second pair of support arms are coupled to opposing sides of the cape.

40. The method of claim 34, wherein the first pair of support arms are attached to an Obturator Internus muscle of the pelvic sidewall and the second pair of support arms are attached to a Levator Ani muscle of the pelvic sidewall.

41. The method of claim 34, wherein each of the support arms include a fastener for attaching the respective support arm to the pelvic sidewall.

42. The method of claim 41, wherein each fastener comprises:
a body portion comprising:
an exterior;
a proximal end coupled to one of the plurality of support arms; and
a distal end; and
one or more barbs coupled to the exterior of the body portion for securing the fastener to the pelvic sidewall.

43. The method of claim 42, wherein the one or more barbs extend from the exterior of the body portion towards the proximal end of the body portion.

44. The method of claim 42, wherein the one or more barbs extend from the exterior of the body portion towards the distal end of the body portion.

45. The method of claim 42, further comprising dynamically collapsing and extending the barbs from the exterior of the body portion to allow the fastener to be inserted and removed from the pelvic sidewall.

46. The method of claim 42, wherein the body portion is hollow and the proximal end of the body portion and the distal end of the body portion are open, the method further comprising passing a needle through the body portion into the pelvic sidewall to secure the fastener to the pelvic sidewall.

47. The method of claim 42, further comprising attaching the fastener to the pelvic sidewall using a pointed distal end of the body portion, the pointed distal end for piercing the pelvic sidewall.

48. The method of claim 34, further comprising adjusting the support arms using a tension adjustment system.

49. The method of claim 48, wherein the tension adjustment system comprises a plurality of grommets, wherein each one of the plurality of grommets is coupled to a respective one of the plurality of support arms using a respective one of a plurality of tension sutures.

50. The method of claim 49, further comprising:
sliding each respective tension grommet along the respective tension suture to adjust a level of tension for the respective support arm; and
securing each respective tension grommet using the respective tension suture.

51. The method of claim 48, wherein the tension adjustment system comprises a plurality of fasteners, wherein each one of the plurality of fasteners is coupled to a respective one of the plurality of support arms using a respective one of a plurality of tension sutures.

52. The method of claim 51, wherein each of the plurality of fasteners include a button, wherein each button comprises:
a first portion attached to the respective support arm and comprising one or more openings to allow the passage of the respective tension suture therethrough; and
a second portion releasably attached to the first portion and comprising one or more openings to allow the passage of the respective tension suture therethrough.

53. The method of claim 52, further comprising adjusting a tension level for each support arm, wherein adjusting the tension level comprises:
attaching the first portion of the button to the second portion of the button;
adjusting a level of tension for the respective support arm using the respective tension suture; and
securing each respective button using the respective tension suture.
